(19) United States
(12) Patent Application Publication
(10) Pub. No.: US 2025/0360215 A1
(43) Pub. Date: Nov. 27, 2025

(54) PHARMACEUTICAL COMBINATIONS AND METHODS FOR PREVENTING OR TREATING NEURODEGENERATIVE DISEASES

(71) Applicants: Chiou-Hwa YUH, Rosemead, CA (US); National Health Research Institutes, Miaoli County (TW)

(72) Inventors: Jyh-Lyh JUANG, Miaoli County (TW); Rai-Hua LAI, Miaoli County (TW)

(21) Appl. No.: 19/104,796

(22) PCT Filed: Aug. 18, 2023
(86) PCT No.: PCT/US2023/072527

§ 371 Date: Feb. 19, 2025

Related U.S. Application Data
(60) Provisional application No. 63/373,009, filed on Aug. 19, 2022.

(51) Int. Cl. A61K 45/06; A61K 9/70; A61K 31/426; A61P 25/28

(52) U.S. Cl. CPC A61K 45/06; A61K 9/7023; A61K 31/426; A61P 25/28

(57) ABSTRACT

Provided is a pharmaceutical combination for preventing or treating a neurodegenerative disease. The pharmaceutical combination includes a first agent being an insulin sensitizer and a second agent being a lipid metabolism modulator. Also provided is a method for preventing or treating a neurodegenerative disease in a subject in need thereof by administering the first agent and the second agent to the subject, thereby reducing the visceral adiposity and the accumulation of amyloid β peptides in the subject.

### TECHNICAL FIELD

The present disclosure relates to synergistic compositions of insulin sensitizers and lipid metabolism modulators for ameliorating deposition of amyloid β peptides, and particularly to pharmaceutical combinations and methods for preventing or treating neurodegenerative diseases by administering the pharmaceutical combinations.

### BACKGROUND

Dementia is a general term for any disease that causes a change in memory and/or thinking skills that is severe enough to impair a person's daily functioning (e.g., driving, shopping, balancing a checkbook, working, and communicating). Most types of dementia cause a gradual worsening of symptoms over the course of years due to progressive damage to nerve cells in the brain caused by the underlying disease process, which is referred to as neurodegeneration. Alzheimer's disease (AD), the leading cause of dementia worldwide, is characterized by the accumulation of the amyloid β peptides (Aβ) within the brain and affects ever-larger numbers of individuals in the aging population. The Alzheimer's Disease International reports that there are over 50 million people worldwide living with dementia in 2020, and this number will almost double every 20 years, reaching 82 million in 2030 and 152 million in 2050. Unfortunately, there is currently no cure for most types of dementia.

If there was one valuable lesson learned from numerous clinical trial failures on new Alzheimer's disease drugs, it is that early therapeutic intervention should be taken for the disease when amyloid β (Aβ) deposits and tangles have not yet caused irreversible damage in the brain. In this regard, mild cognitive impairment (MCI) has come to be recognized as an intermediate state of subclinical impairment whereby individuals may have cognitive symptoms that are serious enough to be noticed, but still maintain the ability to independently carry out everyday activities. MCI can be an early stage of the disease continuum including for Alzheimer's disease if the hallmark changes in the brain are present.

Hence, there is a need in the art to develop competent medications that have potential to effectively treat neurodegenerative diseases at stages where it is not clinically expressed and in the early stages of its clinical expression.

### SUMMARY

In view of the foregoing, the present disclosure provides a pharmaceutical combination for preventing or treating a neurodegenerative disease. Drug combination is a strategy that combines two or more drugs functioned by targeting different drug targets or pathways in overlapping regimens to achieve a synergistically enhanced therapeutic effect while lowering down the dose usage of each individual drug.

In at least one embodiment of the present disclosure, the pharmaceutical combination comprises a first agent being an insulin sensitizer and a second agent being a lipid metabolism modulator. The pharmaceutical combination of the present disclosure comprises at least two drugs targeting different signaling pathways, and can be more effective and less harmful than a single drug therapy.

In at least one embodiment of the present disclosure, the insulin sensitizer used as the first agent may be a hypoglycemic agent. The examples of the hypoglycemic agent include an agonist of peroxisome proliferator-activated receptor gamma (PPARγ), a sulfonylurea derivative, a biguanide derivative, and a glucosidase inhibitor. In some embodiments, the PPARγ agonist may be a thiazolidinedione derivative, which is selected from the group consisting of pioglitazone, rosiglitazone, troglitazone, lobeglitazone, ciglitazone, darglitazone, englitazone, netoglitazone, rivoglitazone, balaglitazone, and any combination thereof. In some embodiments, the sulfonylurea derivative is selected from the group consisting of glyburide, glibenclamide, glimepiride, chlorpropamide, glipizide, tolazamide, tolbutamide, and any combination thereof. In some embodiments, the biguanide derivative is selected from the group consisting of metformin, phenformin, buformin, and any combination thereof. In some embodiments, the glucosidase inhibitor is selected from the group consisting of acarbose, miglitol, voglibose, and any combination thereof.

In at least one embodiment of the present disclosure, the second agent is an agonist of thyroid hormone receptor. In some embodiments, the agonist of thyroid hormone receptor is selected from the group consisting of triiodothyronine, thyroxine, an agonist of peroxisome proliferator-activated receptor alpha (PPARα), and any combination thereof. In some embodiments, the PPARα agonist is selected from the group consisting of clofibrate, gemfibrozil, ciprofibrate, bezafibrate, fenofibrate, and any combination thereof.

In at least one embodiment of the present disclosure, the pharmaceutical combination is formulated with at least one pharmaceutically acceptable carrier to form a single composition. In some embodiments, the pharmaceutical combination is formulated with at least one pharmaceutically acceptable carrier to form a transdermal patch. In some embodiments, the first agent and the second agent are each formulated with at least one pharmaceutically acceptable carrier to form separate compositions.

In at least one embodiment of the present disclosure, a method for preventing or treating a neurodegenerative disease in a subject in need thereof is provided. The method comprises administering to the subject a therapeutically effective amount of the first agent and the second agent as described above. In some embodiments, the neurodegenerative disease is associated with accumulation of amyloid β peptides in brain of the subject. In some embodiments, the neurodegenerative disease is mild cognitive impairment (MCI), early-stage Alzheimer's disease, vascular dementia, frontotemporal dementia, semantic dementia, or dementia with Lewy bodies.

In at least one embodiment of the present disclosure, the first agent and the second agent are administered simultaneously or sequentially. In at least one embodiment of the present disclosure, the first agent and the second agent are administered transdermally.

In at least one embodiment of the present disclosure, the combined administration of the first agent and the second agent has a synergistic effect in reducing the accumulation of the amyloid β peptides in the brain of the subject and/or reducing visceral adiposity in the subject, thereby effectively preventing or treating the neurodegenerative disease.

In the present disclosure, with the regulation of lipid and glucose homeostasis, the pharmaceutical combination and the method provided herein may be used to efficiently reduce visceral adiposity and Aβ accumulation, and thus may be useful for preventing or delaying the progression of neurodegenerative diseases, such as MCI or early-stage AD.

### BRIEF DESCRIPTION OF THE DRAWINGS

For a full understanding of this disclosure, reference should be made to the following detailed descriptions, taken in connection with the accompanying drawings.

FIGS. 1A to 1F show the increased fat deposition in AD mice. FIG. 1A shows the body weight changes of APP/PS1 mice (AD, n=6) and wild-type mice (WT, n=6) at 2- to 6-month-old. FIG. 1B shows the representative micro-computed tomography (CT) imaging and the percentage of body fat of wild-type (WT) mice and APP/PS1 (AD) mice at 5 months of age. * p<0.05. FIG. 1C shows the representative image of gonadal white adipose tissues (gWAT) in AD mice and age-match WT mice and the quantification thereof, in which the upper panel shows gWAT in WT mice and AD mice at 5 months of age, and the lower panel shows sections of gWAT that are stained with hematoxylin and eosin (H&E). Scale bar: 20 μm. *** p<0.01. FIG. 1D shows the representative image of brown adipose tissue (BAT) and interscapular WAT (iWAT) in AD mice and age-match WT mice and the quantification thereof, in which the upper panel shows adipose tissues in WT mice and AD mice at 5 months of age, and the lower panel shows sections of BAT that are stained with hematoxylin and eosin (H&E). Scale bar: 20 μm. ** p=0.01. FIG. 1E shows the Western blot of uncoupling protein 1 (UCP1), cytochrome c oxidase subunit IV (COXIV), and voltage-dependent anion channel (VDAC) levels in WT mice (n=3) and AD mice (n=3). Tubulin: internal control. FIG. 1F shows the representative immunofluorescent micrographs of UCP1 in BAT of WT mice and AD mice. Scale bar: 20 μm. DAPI: 4′,6-diamidino-2-phenylindole.

FIGS. 2A to 2G show the impaired glucose metabolism and inflammatory response associated with visceral adiposity in AD mice. FIG. 2A shows the result of quantitative real-time reverse transcription-polymerase chain reaction (qRT-PCR) analysis on relative ApoE and leptin gene expressions in gWAT of wild-type (WT) mice and APP/PS1 (AD) mice. FIG. 2B shows the result of enzyme-linked immunosorbent assay (ELISA) on the levels of glucose, leptin, and adiponectin (ADP) in serum of WT mice and AD mice. * p<0.05; ** p<0.01. FIG. 2C shows the representative immunofluorescent micrographs of CD11c in gWAT of WT mice and AD mice and the quantification thereof. Sections of gWAT are stained with CD11c and DAPI for M1 macrophage. Scale bar: 20 μm. *** p<0.001. FIG. 2D shows the result of Western blot (WB) on monocyte chemoattractant protein-1 (MCP1) level in gWAT lysate of WT mice (n=3) and AD mice (n=3). Ponceau S: internal control. FIG. 2E shows the result of ELISA on the MCP1 level in serum of WT mice (n=5) and AD mice (n=5). ** p=0.02 by unpaired t-test. FIGS. 2F and 2G show the results of qRT-PCR analysis on the expressions of inflammatory genes (TNFα and INFγ in FIG. 2F) and anti-inflammatory genes (CD206, IL10, Arg1 and YM1 in FIG. 2G) in gWAT of WT mice and AD mice.

FIGS. 3A to 3E show the increased lipid deposition induced by Aβ42. FIG. 3A shows the body weight changes of wild-type (WT) mice with or without Aβ intraperitoneal (IP) injection, in which 4-month-old wild-type mice are injected with three times Aβ42 (30 μg, n=5) or PBS (n=5) only for every 3 days. * p=0.01 by unpaired t-test. FIG. 3B shows the hematoxylin and eosin (H&E) staining of gWAT in wild-type mice with or without Aβ42 injection (n=5). ** p=0.01 by unpaired t-test. FIG. 3C shows that Aβ42 increases lipid accumulation in 3T3-L1 cells, in which 3T3-L1 cells are treated with 1 μM Aβ42, 1 μM Aβ40, or dimethyl sulfoxide (DMSO) as control group (Ctrl) for 24 hours and the lipid droplets therein are examined by Nanolive 3D Cell Explorer. FIG. 3D shows the HCS LipidTOX Red neutral lipid stain of 3T3-L1 cells treated with 1 μM Aβ42, 1 μM Aβ40, or DMSO (Ctrl). FIG. 3E shows the dose-dependent increase of ApoE gene expression in 3T3-L1 cells induced by Aβ42, as measured by qRT-PCR.

FIGS. 4A to 4H show that Aβ42 increases fat deposition through liver X receptor (LXR) signaling. FIGS. 4A and 4B show that Aβ42 dysregulates liver X receptor/retinoid X receptor (LXR/RXR) signaling in gWAT of AD mice (FIG. 4A) or in 3T3-L1 cells treated with 1 μM Aβ42 or 1 μM Aβ40 (FIG. 4B), as demonstrated by Ingenuity Pathway Analysis (IPA). The upper panel shows IPA-based enrichment of biological functions, and the lower panel shows the heatmap involved in LXR/RXR activation across this analysis. FIG. 4C shows the significant functional gene sets differentially expressed in 3T3-L1 cells after Aβ42 or Aβ40 treatment, including fatty acid metabolism and lipoprotein biosynthetic process, as demonstrated by Gene Set Enrichment Analysis (GSEA). FIG. 4D shows the result of Western blot of LXR signaling proteins in 3T3-L1 cells, in which the detergent-resistant membranes (DRM) fractions lysate or conditioned medium of Aβ42-treated 3T3-L1 cells are examined. FIGS. 4E and 4H show the result of Western blot of LXR signaling proteins in gWAT (FIG. 4E) or BAT (FIG. 4H) of wild-type (WT) mice and APP/PS1 (AD) mice (n=2). FIG. 4F shows the representative immunofluorescent micrographs of ABCA1 and caveolin-1 in gWAT of AD mice and the quantification thereof. FIG. 4G shows the plasma high-density lipoprotein/total cholesterol (HDL/T-Chol) level in WT mice and AD mice (n=5 or 6). ** p<0.01. ABCA1: ATP binding cassette subfamily A member 1. PPAγ: peroxisome proliferator-activated receptor gamma. Actin, GAPDH (glyceraldehyde-3-phosphate dehydrogenase) and tubulin: internal controls, respectively.

FIG. 5 shows that the UCP1 and PPARγ2 levels were decreased and increased, respectively, upon the treatment of Aβ42 in the MDI (methylisobutylxanthine, dexamethasone, insulin)-induced differentiated adipocytes (3T3-L1). * p<0.05 by unpaired t-test.

FIGS. 6A to 6L show that the combination treatment (T+P) of thyroxine (T) and pioglitazone (P) reduces fat deposition and the Alzheimer's pathogenesis. FIG. 6A shows the result of ELISA on the levels of free T4 (fT4) and thyroid-stimulating hormone (TSH) in serum of WT mice and AD mice. * p<0.05; ** p<0.01. FIG. 6B shows the UCP1 and PPARγ2 levels in Aβ42-treated 3T3-L1 cells with or without thyroxine (T4) and/or pioglitazone (pio), in which Aβ42-treated 3T3-L1 cells are treated with MDI (methylisobutylxanthine, dexamethasone, insulin) induction medium and with 100 nM thyroxine (T4) and/or 10 μM pioglitazone (pio) indicated. *** p<0.001 by unpaired t-test. FIG. 6C shows that T4 and pioglitazone reduce the increase of lipid accumulation in Aβ42-treated 3T3-L1 cells, in which the Aβ42-treated 3T3-L1 cells are treated with 100 nM thyroxine and 10 μM pioglitazone for 24 hours before assay. FIG. 6D shows the result of Western blot on the UCP1 level in gWAT and BAT of APP/PS1 mice with or without thyroxine and pioglitazone (n=2 to 5). FIG. 6E shows the result of Western blot on the LXR signaling proteins in gWAT of APP/PS1 mice with or without thyroxine and pioglitazone. FIG. 6F shows representative immunofluorescent micrographs of CD11c in gWAT of APP/PS1 mice with or without thyroxine and pioglitazone, in which sections of gWAT are stained with CD11c and DAPI for M1 macrophage. Scale bar: 20 μm. FIG. 6G shows the result of Western blot on the MCP1 level in gWAT of APP/PS1 mice with or without thyroxine and pioglitazone. FIGS. 6H and 6I show the HDL/T-Chol (FIG. 6H) and glucose (FIG. 6I) levels in plasma of APP/PS1 mice with or without thyroxine and pioglitazone. ** p<0.01. FIG. 6J shows the result of Western blot on the Aβ42 (Aβ) and glial fibrillary acidic protein (GFAP) levels in the hippocampus of wild-type (WT) mice and APP/PS1 (AD) mice with or without thyroxine and pioglitazone. FIG. 6K shows the representative immunofluorescent micrographs of gliosis and amyloid aggregates in brain tissues of APP/PS1 mice with or without thyroxine (T4) and pioglitazone (pio). FIG. 6L shows the result of Western blot on the levels of β-secretase (BACE), nicastrin, p62, and microtubule-associated protein 1A/1B-light chain 3 (LC3) in the hippocampus of APP/PS1 (AD) mice with or without thyroxine and pioglitazone. Tubulin, GAPDH (glyceraldehyde-3-phosphate dehydrogenase), and ponceau S: internal controls, respectively.

### DETAILED DESCRIPTION

The description discloses some embodiments in such a detail that a person skilled in the art is able to utilize the embodiments based on the disclosure. Not all steps or features of the embodiments are discussed in detail, as many of the steps or features will be obvious for a person skilled in the art based on this disclosure.

As used in this disclosure, the singular forms “a,” “an,” and “the” include plural referents unless the content clearly dictates otherwise. As used herein, the term “and” is intended to be inclusive unless otherwise indicated. As used herein, the term “or” is generally employed in its sense including “and/or” unless the context clearly dictates otherwise.

As used herein, the term “about” generally means within 10%, 5%, 1%, or 0.5% of a given value or range. Alternatively, the term “about” means within an acceptable standard error of the mean when considered by one of ordinary skill in the art. Unless otherwise expressly specified, all of the numerical ranges, amounts, values and percentages such as those for quantities of materials, durations of time periods, temperatures, operating conditions, ratios of amounts, and the likes disclosed herein should be understood as modified in all instances by the term “about.”

As used herein, the terms “comprising,” “having,” “including,” and “containing” are to be construed as open-ended terms (i.e., meaning “including, but not limited to”) unless otherwise noted. For example, a composition, mixture, process or method that comprises a list of elements or actions is not necessarily limited to only those elements or actions, but may include other elements or actions not expressly listed, or inherent to such composition, mixture, process, or method.

It is understood that, as used herein, the identifiers “first” and “second” are used solely to aid in distinguishing the various components and steps of the disclosed subject matter. The identifiers “first” and “second” are not intended to imply any particular order, amount, preference, or importance to the components or steps modified by these terms.

In at least one embodiment, the present disclosure is directed to a pharmaceutical combination for preventing or treating a neurodegenerative disease, comprising a combined use of an insulin sensitizer as a first agent and a lipid metabolism modulator as a second agent. In at least one embodiment, the present disclosure is directed to a method for preventing or treating a neurodegenerative disease in a subject in need thereof by administering the pharmaceutical combination in a therapeutically effective amount to the subject.

As used herein, the term “prophylactic,” “preventing” or “prevention” refers to preventive or avoidance measures for a disease or the symptoms or conditions thereof, which include but are not limited to applying or administering one or more active agents to a subject who has not yet been diagnosed as a patient suffering from the disease or the symptoms or conditions thereof but may be susceptible or prone to the disease, e.g., a neurodegenerative disease in this disclosure. The purpose of the preventive measures is to avoid, prevent, or postpone the occurrence of the disease or the symptoms or conditions thereof.

As used herein, the term “treating” or “treatment” refers to obtaining a desirable pharmacologic and/or physiologic effect, e.g., ameliorating Aβ deposition. The effect may be prophylactic in terms of completely or partially preventing a disease or the symptoms or conditions thereof and/or therapeutic in terms of completely or partially curing, alleviating, relieving, remedying, or ameliorating a disease or an adverse effect attributable to the disease or the symptoms or conditions thereof.

As used herein, the terms “patient,” “individual,” “host” and “subject” are used interchangeably. The term “subject” means a human or an animal. Examples of the subject include, but are not limited to, human, monkey, mice, rat, woodchuck, ferret, rabbit, hamster, cow, horse, pig, deer, dog, cat, fox, wolf, chicken, emu, ostrich, and fish. In some embodiments, the subject is a mammal, e.g., a primate such as a human.

As used herein, the phrase “a therapeutically effective amount” refers to the amount of an active agent that is required to confer a desired therapeutic effect on a subject in need thereof. Effective doses may vary, as recognized by those skilled in the art, depending on routes of administration, excipient usage, the possibility of co-usage with other therapeutic treatment, and the condition to be treated.

In some embodiments, the first agent (e.g., a thiazolidinedione derivative) used in the present disclosure is administrated in a therapeutically effective amount of about 0.01 mg to about 100 mg (e.g., about 0.01 mg to about 100 mg, about 0.01 mg to about 90 mg, about 0.01 mg to about 80 mg, about 0.02 mg to about 70 mg, about 0.05 mg to about 60 mg, about 0.1 mg to about 50 mg, about 0.5 mg to about 40 mg, about 1 mg to about 30 mg, about 5 mg to about 20 mg, and about 7.5 mg to about 10 mg) per dose. In some embodiments, the second agent (e.g., an agonist of the thyroid hormone receptor) used in the present disclosure is administrated in a therapeutically effective amount of about 0.01 g to about 200 g (e.g., about 0.01 μg to about 200 μg, about 0.01 μg to about 150 g, about 0.01 μg to about 125 μg, about 0.02 μg to about 100 μg, about 0.05 μg to about 75 μg, about 0.1 μg to about 60 μg, about 0.5 μg to about 50 μg, about 1 μg to about 45 μg, about 5 μg to about 30 μg, and about 7.5 μg to about 15 μg) per dose.

In at least one embodiment, the weight ratio of the therapeutically effective amount of the first agent to the therapeutically effective amount of the second agent may be from 1,000,000:1 to 1:20. In some embodiments, the weight ratio of the first agent and the second agent used in the present disclosure may be about 1,000,000:1, 500,000:1, 100,000:1, 50,000:1, 10,000:1, 5,000:1, 1,000:1, 900:1, 800:1, 700:1, 600:1, 500:1, 400:1, 300:1, 200:1, 100:1, 75:1, 50:1, 25:1, 20:1, 10:1, 5:1, 2:1, 1:1, 1:2, 1:5, 1:10, 1:15, or 1:20, where any value can be a lower and upper end-point of a range. In some embodiments, the desired ratio of the first agent to the second agent may depend on the type and stage of the neurodegenerative disease being treated in the subject. For example, the weight ratio of the first agent (e.g., a thiazolidinedione derivative) to the second agent (e.g., an agonist of the thyroid hormone receptor) can be increased in the progress of Alzheimer's disease.

As used herein, the term “administering” or “administration” refers to the placement of an active agent into a subject by a method or route which results in at least partial localization of the active agent at a desired site to produce the desired effect. The active agent described herein may be administered by any appropriate route known in the art. In some embodiments, the first agent and the second agent used in the present disclosure are formulated for oral, subcutaneous, intravenous, transdermal, intraperitoneal, intramuscular, intracerebroventricular, intraparenchymal, intrathecal, intracranial, buccal, mucosal, nasal, or rectal administration.

In at least one embodiment, the first agent and the second agent are transdermally administered to the subject. In some embodiments, the first agent and the second agent may be administered by a transdermal patch, which comprises a backing layer, a removable release liner, and an adhesive drug layer positioned between the backing layer and the release liner, wherein the adhesive drug layer is composed of an adhesive matrix comprising at least one of the first agent and the second agent used herein. In some embodiments, when administration, the release liner is removed, such that the adhesive drug layer can adhere to the skin of the subject at the administration site. In some embodiments, the adhesive matrix serves to release the first and second agents to the skin as well as secure the patch to the skin. In some embodiments, the transdermal patch is useful for prolonged or long-term delivery of the first and second agents.

In at least one embodiment, the first agent and the second agent used in the present disclosure may be optionally formulated with one or more pharmaceutically acceptable carriers. In some embodiments, the first agent and the second agent are formulated into a single composition, and they may be administered simultaneously in the single composition. In some embodiments, the first agent and the second agent are each formulated into separate compositions, and they may be administered simultaneously or sequentially in the different compositions. In some embodiments, the first agent and the second agent are formulated with at least one pharmaceutically acceptable carrier to form a transdermal patch.

As used herein, the term “pharmaceutically acceptable carrier” refers to a pharmaceutically acceptable material, composition, or vehicle, such as diluents, disintegrates, binders, adhesives, lubricants, glidants, and surfactants, which does not abrogate the biological activity or properties of the active agent, and is relatively non-toxic; that is, the material may be administered to an individual without causing an undesirable biological effect or interacting in a deleterious manner with any of the components of the composition in which it is contained.

As used herein, the term “synergistic” refers to a combination of therapies which is more effective than those of single therapies.

As used herein, the term “neurodegenerative disease” refers to a condition related to the death of neurons in different regions of the nervous system and the consequent functional impairment of an affected subject. The neurodegenerative disease may encompass mild cognitive impairment (MCI), Alzheimer's disease (AD) such as early-stage Alzheimer's disease, vascular dementia, frontotemporal dementia, semantic dementia, and/or dementia with Lewy bodies.

Many examples have been used to illustrate the present disclosure. The examples below should not be taken as a limit to the scope of the present disclosure.

### EXAMPLES

Materials and methods

The materials and methods used in the following Examples 1-6 were described in detail below. The materials used in the present disclosure but unannotated herein were commercially available.

### (1) Mice

Double transgenic APP/PS1 (amyloid precursor protein/presenilin-1) mice were purchased from Jackson Laboratory (Bar Harbor, ME, USA) to breed with wild-type (WT) B6C3F1 (C57BL/6N background) mice.

Serum adiponectin, leptin, monocyte chemoattractant protein-1 (MCP1), free T4, and thyroid-stimulating hormone (TSH) levels in APP/PS1 and WT mice were determined by enzyme-linked immunosorbent assay (ELISA) kit (Elabscience) at the indicated time points.

All experimental animal procedures and protocols were approved by the Institutional Animal Care and Use Committee at National Health Research Institutes (NHRI) (approved protocol No. NHRI-IACUC-1080070-A).

### (2) Micro-Computed Tomography (CT) Imaging

Male APP/PS1 and aged-match control mice were imaged at age of 5 months (n=3/group). Whole-body composition analysis was conducted with micro-CT imaging using a Skyscan 1076 High-resolution X-ray micro-CT system by Taiwan Mouse Clinic.

### (3) Cell Culture

The 3T3-L1 pre-adipocytes (human neuroblastoma, ATCC CL-173) were cultured in Dulbecco's modified Eagle medium (DMEM) (Invitrogen, USA). Cells were grown at 37° C. in a 5% CO₂ humid atmosphere. The differentiation of 3T3-L1 cells into adipocyte-like cells was performed as follows. A confluence of 70% of 3T3-L1 cells was induced with MDI induction medium (containing 500 μM 3-isobutyl-1-methylxanthine (IBMX), 1 μg/mL insulin, and 1 μM dexamethasone) for 3 days, followed by changing the medium to differentiation medium (10% fetal bovine serum (FBS) in DMEM). The differentiation medium was refreshed every two days. Full differentiation was achieved by day 8.

### (4) Tissue Sectioning and Histology

The white adipose tissue (WAT) and the brown adipose tissue (BAT) were fixed in 4% formalin, embedded in paraffin, sectioned, and stained with hematoxylin and eosin (H&E). All photomicrographs were generated on an Olympus microscope. Representative images were taken with Olympus DP73 camera with cellSens Dimension software for adjusting brightness and contrast and image cropping.

### (5) Microarray and Ingenuity Pathway Analysis (IPA) and Gene Set Enrichment Analysis (GSEA) Gene Sets Analysis

The microarray of mouse Clariom S Assays (Thermo Fisher Scientific) for whole-transcript expression analysis were used to analyze the signaling pathways that were impacted. Ingenuity Pathway Analysis (IPA) was performed for the leading pathway analysis application and Gene Set Enrichment Analysis (GSEA) was performed for the interpreting gene expression data.

### (6) Immunofluorescence

For immunofluorescence, unstained slides were deparaffinized, followed by antigen retrieval using 1× saline-sodium citrate buffer (SSC, a mixture of 150 μM sodium chloride and 15 mM trisodium citrate, pH 6.0) with 0.05% Tween 20. The slides were then blocked with 1% bovine serum albumin (BSA) and 0.05% Tween 20 in phosphate-buffered saline (PBS) for 1 hour, before applying primary antibody overnight. After overnight incubation, the secondary antibody and 4′,6-diamidino-2-phenylindole (DAPI) were applied for 1 hour before washed. The stains were reviewed using an Olympus microscope with an Olympus DP73 camera.

(7) Quantitative Real-Time Reverse Transcription-polymerase Chain Reaction (qRT-PCR)

Total RNAs from brain tissues or culture cells were extracted using the illustra RNAspin Mini RNA Isolation Kit (GE Healthcare Life Sciences) for reverse transcription with the High-Capacity cDNA Reverse Transcription Kits (ABI Applied Biosystems, USA) according to the manufacturer's instructions. The quantitative real-time reverse transcription-PCR analysis was performed using the Fast SYBR Green Master Mix (ABI Applied Biosystems, USA). Results were determined using respective standard curves calculations.

### (8) Quantification and Statistical Analysis

Prism 6 software (GraphPad) was used to analyze data by two-tailed unpaired Student's t-test. When multiple groups were compared, one-way analysis of variance (ANOVA) with the Tukey's test was performed. Data were presented as mean±SEM.

### Example 1: Increased Adiposity in Early-Stage Alzheimer's Disease

To determine the fat-brain axis in Alzheimer's disease, the body weight of the APP/PS1 male mice was observed and analyzed in this example. The APP/PS1 mice were double transgenic mice expressing a chimeric mouse/human amyloid precursor protein and a mutant human presenilin 1, in which both mutations were associated with early-onset Alzheimer's disease.

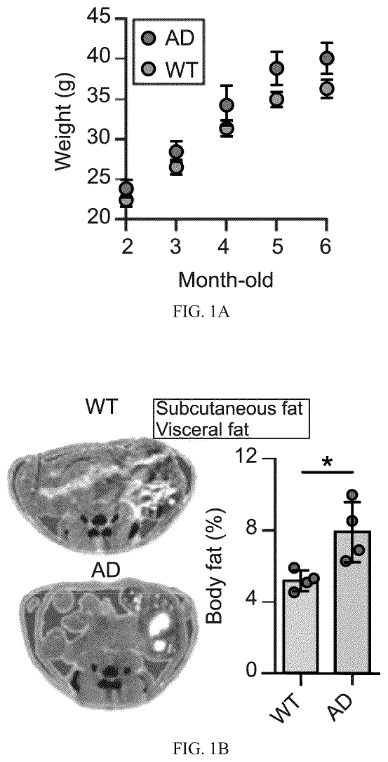

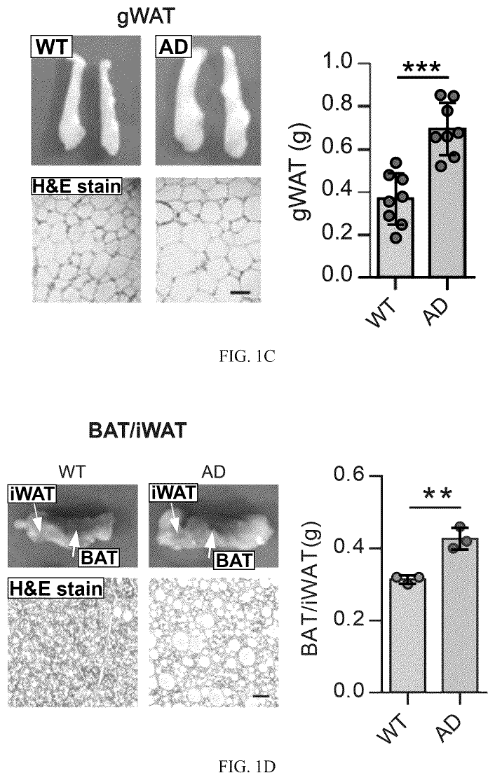

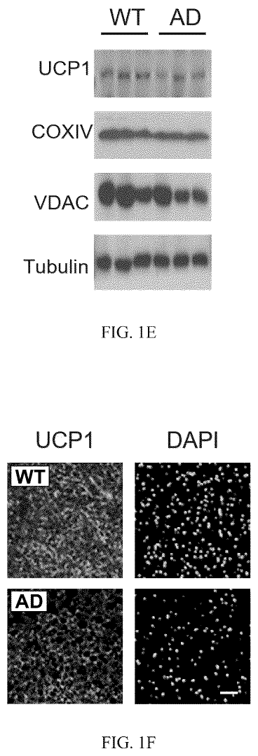

As shown in FIG. 1A, the average body weight of the APP/PS1 mice (AD) was significantly higher than that of the wild-type mice (WT) at 4-6 months of ages, i.e., before Aβ deposits were notably appeared in the brain.

Since the weight change was correlated with visceral fat, the visceral adipose tissue in the APP/PS1 mice was measured. The analysis of micro-CT image showed that the regional body fat deposits were obviously increased in the APP/PS1 mice (AD) compared to the wild-type mice (WT) (FIG. 1B). Further, histological analysis also revealed hypertrophic adipocytes with large and unilocular lipid droplets in the gonadal white adipose tissues (gWAT) of the AD mice compared to the WT mice (FIG. 1C).

In addition to the excessive fat accumulation in WAT, it was also observed that the brown adipose tissues (BAT) of the AD mice were whitening and enlarged with a significant increase in the size of adipocytes (FIG. 1D). Since BAT functions to dissipate energy through uncoupled respiration and heat production, the thermogenic features of the whitening of BAT were further analyzed. Western blot analysis showed decreased levels of uncoupling protein 1 (UCP1) (a thermogenesis mediator), cytochrome c oxidase subunit IV (COXIV) (an electron acceptor of the respiratory chain), and voltage-dependent anion channel (VDAC) (a gatekeeper for mitochondrial energetic flux) as compared to the WT mice (FIGS. 1E and 1F), suggesting a lower thermogenic potential in the BAT of the AD mice.

These results clearly indicated that the APP/PS1 mice consistently exhibited a prevalence of obesity at an early disease stage, implying that the visceral adipose tissue was one feature of early-stage AD.

### Example 2: Decline of Glucose Metabolism in Early-Stage Alzheimer's Disease

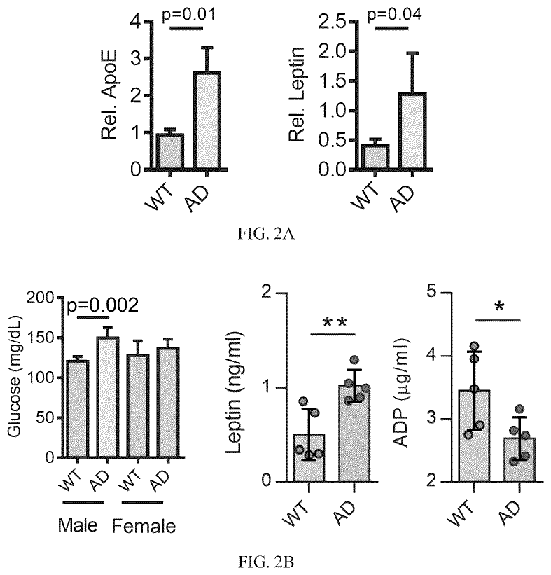

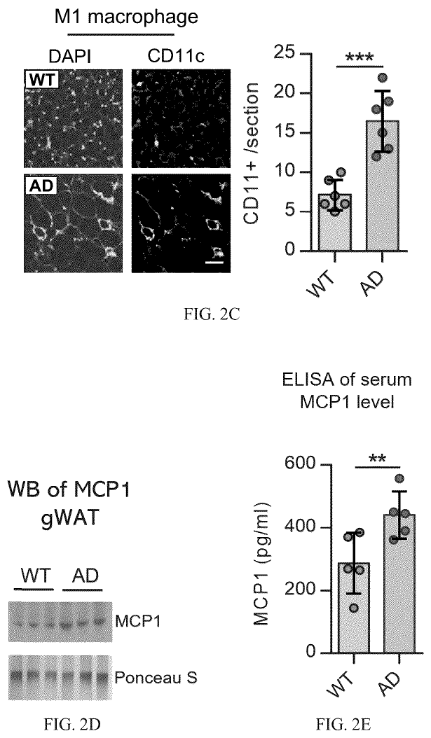

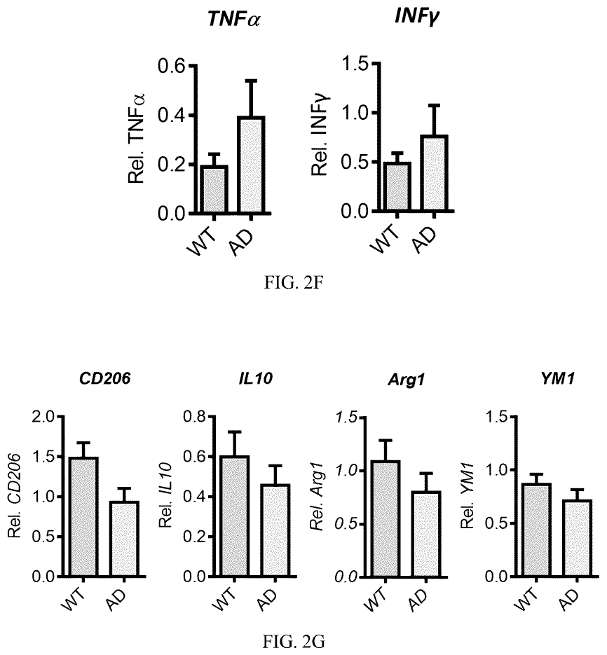

To assess the association of metabolic parameters and adiposity, the level of ApoE, which functions in transporting cholesterol-laden lipid, was first examined. The qRT-PCR analysis showed a marked increase of ApoE and leptin gene expressions in gWAT of AD mice compared to WT mice (FIG. 2A). Next, adiponectin was a secretory peptide by adipocytes, whose reduction was associated with lipodystrophy and insulin resistance, such that the circulating levels of glucose, leptin, and adiponectin (ADP) were measured. As shown in FIG. 2B, the AD mice exhibited increased blood glucose and leptin levels, and decreased adiponectin level compared to WT mice, implying that a decline in glucose metabolism was associated with visceral adiposity in early-stage AD.

Further, because visceral obesity was often accompanied by chronic low-grade inflammation^{[1]}, the pro-inflammatory characteristics in gWAT of AD mice were examined. The results showed that the gWAT of AD mice exhibited increased levels of M1 macrophage infiltration and pro-inflammatory MCP-1 chemokine compared to control mice (FIGS. 2C to 2E). Furthermore, the expressions of pro-inflammatory genes, TNFα and INFγ, were also increased (FIG. 2F), and anti-inflammatory gene expressions, CD206, IL10, Arg1 and YM1, were concurrently decreased in the AD visceral tissues compared to WT mice (FIG. 2G).

These results indicated that the impaired glucose metabolism and pro-inflammatory response were involved in adiposity of early-stage AD.

### Example 3: Induction of Lipogenesis in Adipose Tissue by Aβ peptides

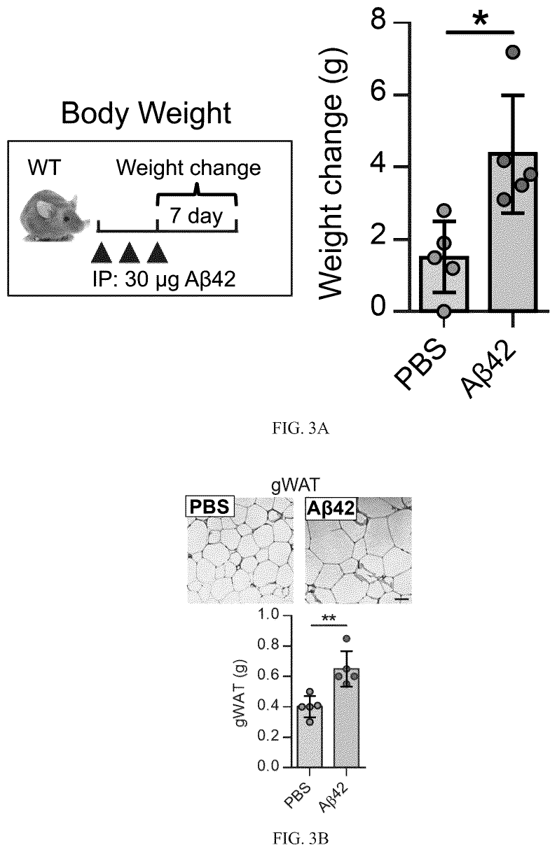

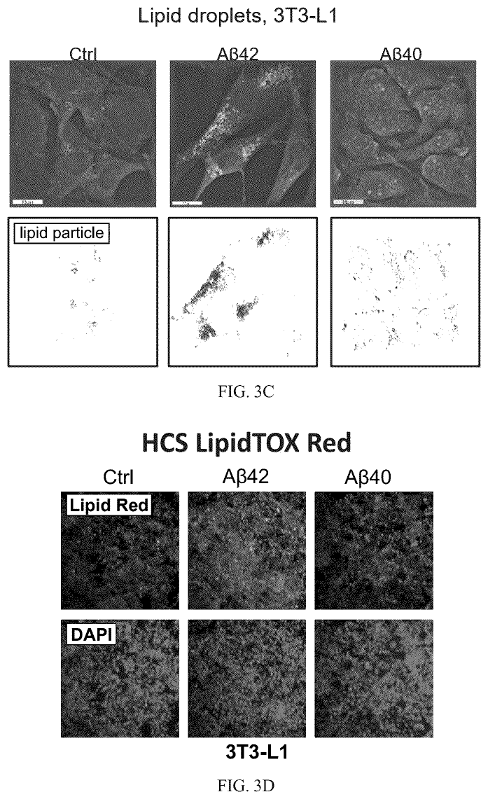

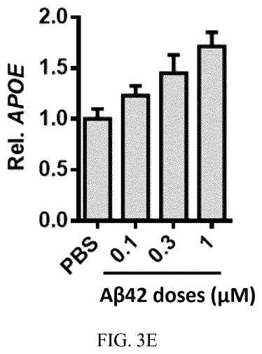

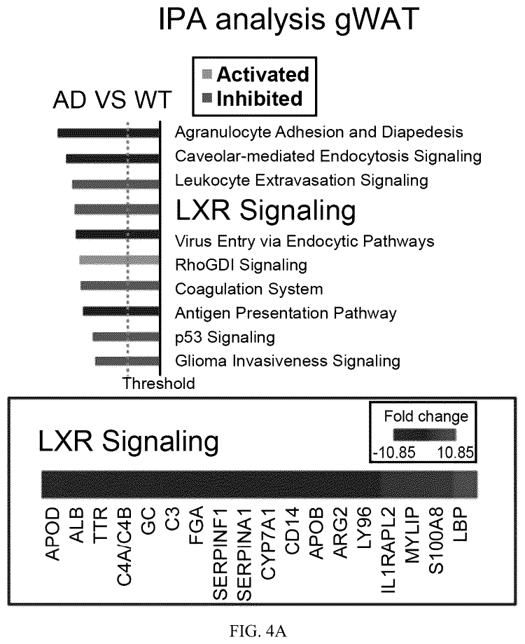

To assess whether the Aβ peptides were involved in lipid deposition in adipose tissues, Aβ42 (30 μg) was injected intraperitoneally into WT mice three times at an interval of 3 days. The results showed that after 7 days of the last injection, the WT mice's body weight and gWAT mass rapidly increased (FIG. 3A), and the increased visceral adiposity was characterized by enlarged hypertrophic adipocytes (FIG. 3B), suggesting that Aβ exerted an obesogenic effect in wild-type mice.

To exclude the possibility that the visceral adiposity was a secondary event caused by factors released from other tissues/organs, 3T3-L1 adipocytes were incubated with Aβ40 or Aβ42 to determine whether and which Aβ peptides to be potent in causing lipid accumulation in adipocytes. By analyzing cell's inner structure by three-dimensional imaging and HCS LipidTOX Red neutral lipid stain, Aβ42, but not Aβ40, evidently stimulated lipid droplet accumulation in adipocytes (FIGS. 3C and 3D). The apoE gene expression was also markedly upregulated by Aβ42 treatment (FIG. 3E). These results indicated that Aβ42 was a direct inducer of lipid storage in adipocytes.

### Example 4: Adiposity induced by Aβ Peptides through Liver X Receptor (LXR) Pathway

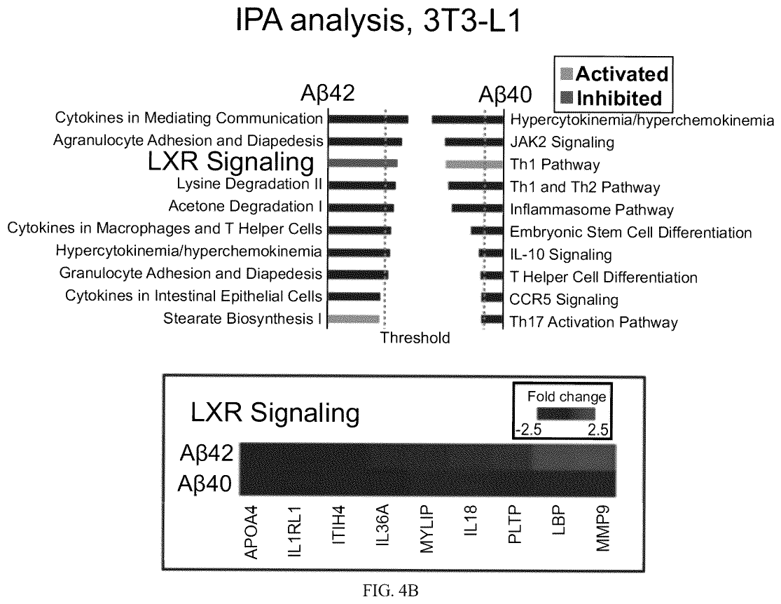

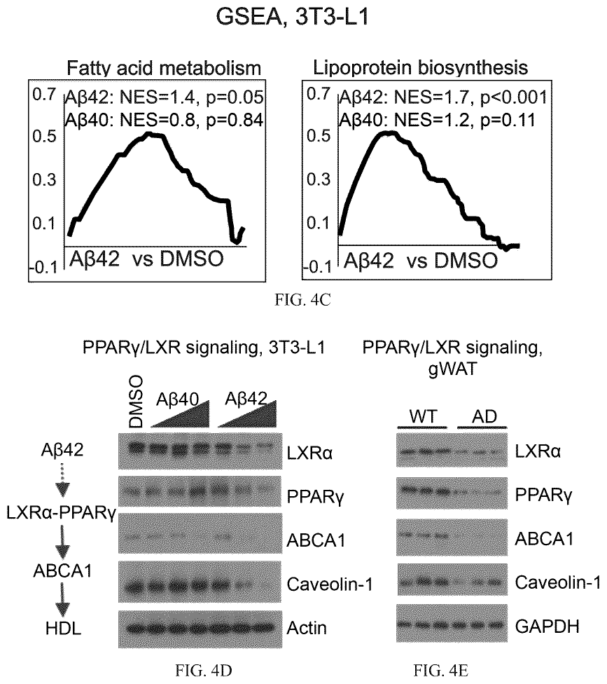

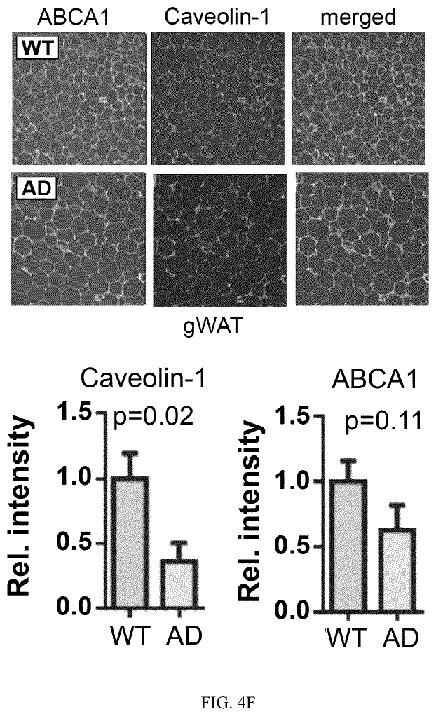

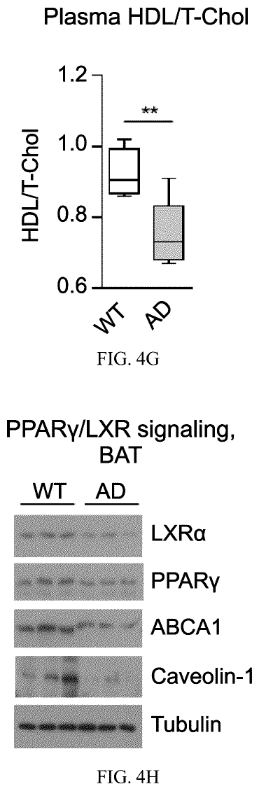

To explore the signaling pathways by which Aβ42 controlled lipid deposition in adipocytes, transcriptional profiling on the Aβ-induced adiposity of gWAT and the 3T3-L1 cells treated with Aβ42 was performed. The Ingenuity Pathway Analysis (IPA) of microarray data suggested that the liver X receptor (LXR) signaling, a key lipogenic nuclear receptor in maintaining lipid homeostasis, was among the top pathways identified in both gWAT of AD mice and the 3T3-L1 cells exposed to AB42 (FIGS. 4A and 4B). Moreover, Gene Set Enrichment Analysis also suggested that the pathways involved in fatty acid metabolism and lipoprotein biosynthesis were altered by Aβ42, further supporting the role of Aβ42 in lipid metabolism (FIG. 4C).

Further, FIG. 4D showed that the treatment of 3T3-L1 cells with Aβ42, but not with Aβ40, markedly decreased protein levels of LXRα, PPARγ (peroxisome proliferator-activated receptor gamma), ABCA1 (ATP binding cassette subfamily A member 1), and caveolin-1. Similarly, the levels of LXRα, PPARγ, ABCA1, and caveolin-1 in gWAT of AD mice were found downregulated compared to WT mice (FIGS. 4E and 4F).

Additionally, as serum high-density lipoprotein (HDL) level was modulated by LXR activity, it was observed that the AD mice exhibited decreased levels of plasma HDL compared to WT mice (FIG. 4G). Given that the BAT in AD has acquired a white-like appearance, it was also investigated whether there was a similar change in LXR signaling activity in BAT. As shown in FIG. 4H, the Western blot showed that the levels in LXRα, PPARγ, ABCA1, and caveolin-1 were decreased in the BAT of AD mice compared to WT mice.

These results indicated that the Aβ42 induced adiposity in early-stage AD through impairing the LXR-ABCA1 pathway, and lipodystrophy was an early feature of AD that contributed to the disease pathogenesis.

### Example 5: Identification of Combination Drugs for Effectively Reducing Visceral Adiposity and Ameliorating Aβ42 Deposition in AD Brain

According to Example 4 above, lipid homeostasis was compromised through the LXR pathway in early-stage AD. However, the treatment of LXR agonists for metabolic disorders has already been shown to cause hepatic steatosis and hypertriglyceridemia in clinical trials^{[2,3]}. Hence, a combination therapy aiming at targeting different signaling pathways associated with LXR was identified in this example.

First, according to FIG. 5, it was found that the PPARγ2 and UCP1 levels were increased and decreased, respectively, in the MDI-induced differentiated 3T3-L1 adipocytes treated with Aβ42. Accordingly, it was assumed that using a combination screen aiming to currently reduce the PPARγ2 level and increase the UCP1 level might ameliorate the disease progression of AD. Therefore, the combination use of four classes of insulin sensitizers, which include pioglitazone (a thiazolidinedione derivative), glimepiride (a sulfonylurea derivative), metformin (a biguanide derivative), and acarbose (a glucosidase inhibitor), and three anti-cholesterol drugs, which include simvastatin (a statin drug), fenofibrate (a derivative of fibric acid), and levothyroxine (a thyroid hormone) were used for screening of combination drugs. These combination drugs were also shown in Table 1 below.

TABLE 1
The anti-diabetes drugs and the anti-cholesterol drugs
used for screening of combination drugs in this Example

| Anti-diabetes drugs | Anti-cholesterol drugs |
|---|---|
| Pioglitazone (thiazolinedione) | Simvastatin (statin) |
| Glimepiride (sulfonylurea) | Fenofibrate (fibric acid) |
| Metformin (biguanide) | Levothyroxine (thyroid hormone) |
| Acarbose (glucosidase inhibitor) | |

The results were reported in Table 2 below, and showed that the Aβ42-treated 3T3-L1 cells exhibited the most significant reduction in PPARγ2 level and increase in UCP1 level upon the combination treatment of pioglitazone and levothyroxine as compared to other combination treatments.

TABLE 2
The expression patterns in the UCP1 and PPARγ2 levels of different
combination drugs in the Aβ42-treated 3T3-L1 cells

| Anti-diabetes | Anti-cholesterol | | | |
|---|---|---|---|---|
| drugs | drugs | Condition | PPARγ2 (log2) | UCP1 (log2) |
| — | — | Ctrl | −2.08 ± 0.05 | 0.22 ± 0.4 |
| — | — | MDI | −0.64 ± 0.08 | 0.47 ± 7 |
| — | — | MDI + Aβ42 | 0.0 ± 0.07 | 0.0 ± 0.05 |
| Acarbose | Fenofibrate | MDI + Aβ42 | 0.38 ± 0.06 | 0.30 ± 0.06 |
| | Simvastatin | MDI + Aβ42 | −0.62 ± 0.05 | −0.14 ± 0.04 |
| | Levothyroxine | MDI + Aβ42 | 0.89 ± 0.09 | 0.14 ± 0.07 |
| Glimepiride | Fenofibrate | MDI + Aβ42 | 1.13 ± 0.10 | 0.21 ± 0.0.04 |
| | Simvastatin | MDI + Aβ42 | 0.02 ± 0.03 | 0.21 ± 0.06 |
| | Levothyroxine | MDI + Aβ42 | −0.13 ± 0.04 | −0.34 ± 0.02 |
| Metformin | Fenofibrate | MDI + Aβ42 | 0.96 ± 0.03 | 0.62 ± 0.04 |
| | Simvastatin | MDI + Aβ42 | 0.96 ± 0.05 | 0.78 ± 0.03 |
| | Levothyroxine | MDI + Aβ42 | 1.22 ± 0.07 | 1.08 ± 0.05 |
| Pioglitazone | Fenofibrate | MDI + Aβ42 | −0.64 ± 0.02 | 0.81 ± 0.06 |
| | Simvastatin | MDI + Aβ42 | 0.03 ± 0.03 | −0.05 ± 0.07 |
| | Levothyroxine | MDI + Aβ42 | −1.31 ± 0.07 | 1.23 ± 0.02 |

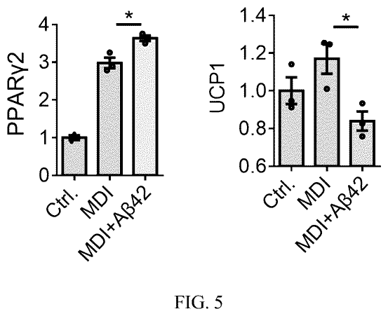

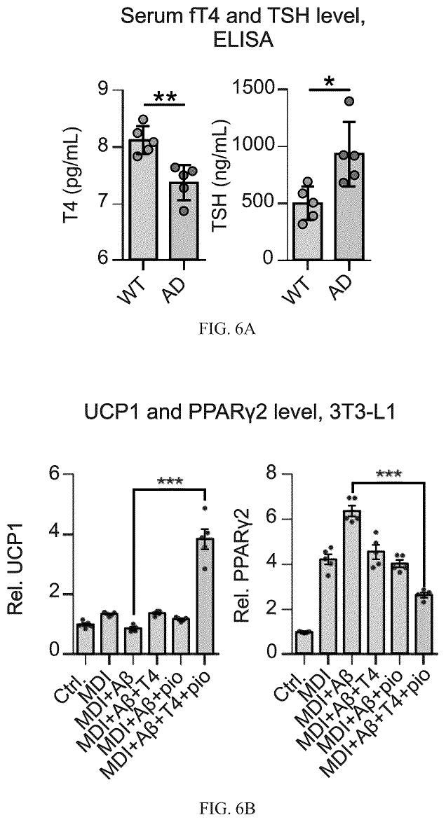

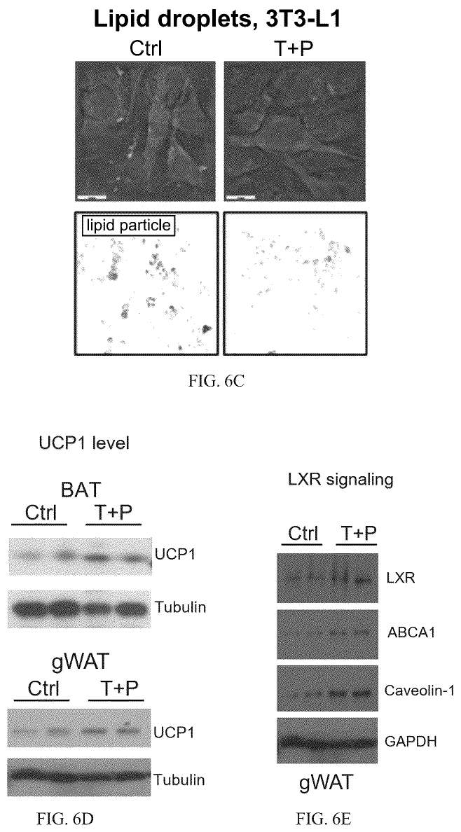

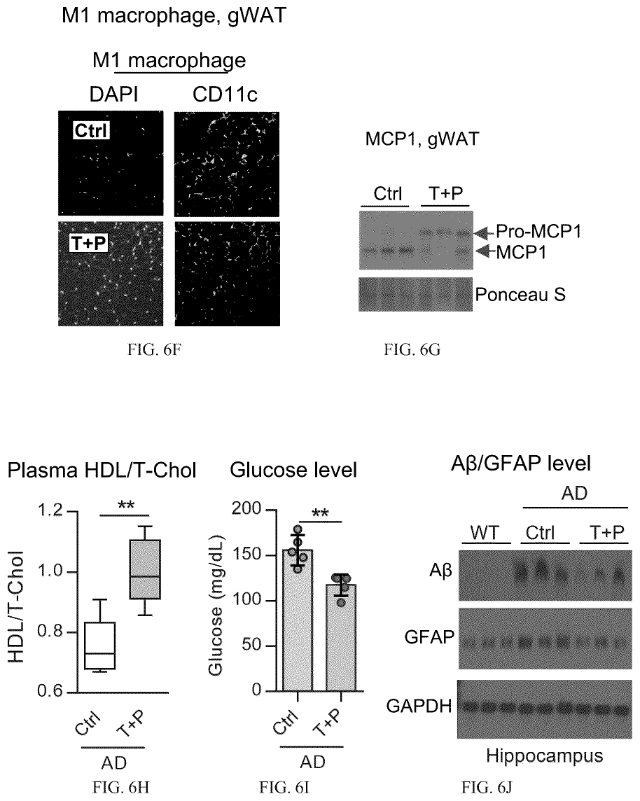

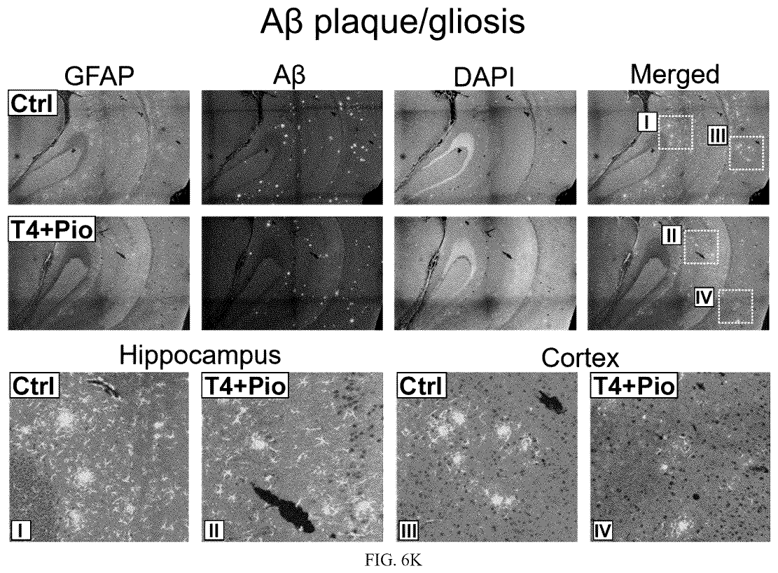

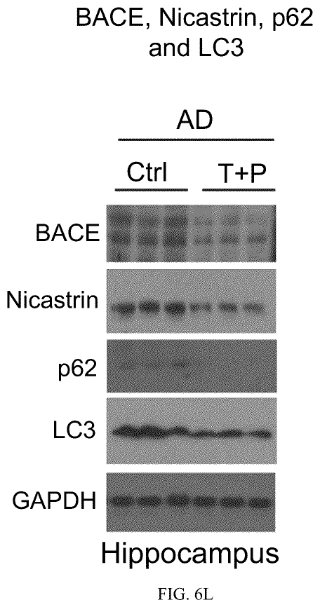

In addition, it was found that the level of free thyroxine (fT4) was significantly decreased in AD mice compared to WT mice, and the AD mice also exhibited significantly higher level of the circulating thyroid-stimulating hormone (TSH) compared to WT mice (FIG. 6A), implying that thyroid hormone homeostasis might be affected by the impaired LXR pathway in early-stage AD.

Therefore, the combination use of thyroid hormone (e.g., T4) and a PPARγ agonist (e.g., pioglitazone) was analyzed, and it was observed that such combined drug treatment markedly increased the UCP1 level and decreased the PPARγ2 level in the Aβ42-treated 3T3-L1 cells compared to mock control or treatment with individual drugs (FIG. 6B). Also, the intracellular lipid droplets were found to be obviously decreased by the combined drug treatment (FIG. 6C). These results suggested that the combination treatment of T4 and pioglitazone effectively reduced fat deposition and increased thermogenesis in adipocytes.

As to the preclinical animal trials, 3-month-old APP/PS1 mice were fed with 0.95 μg thyroxine and 316 μg pioglitazone per day for two months. It was observed that the combined drug treatment markedly increased UCP1 levels in BAT and moderately in gWAT after 2 months of combination therapy (FIG. 6D), suggesting that the impaired thermogenesis in early-stage AD was ameliorated by the combined drug treatment. Further, FIG. 6E showed that the reduced levels of LXR, AβCA1 and caveolin-1 in the gWAT of AD mice were rescued by the combined drug treatment. The increased M1 macrophage infiltration and pro-inflammatory MCP1 levels were also found to be noticeably diminished after the combined drug treatment (FIGS. 6F and 6G). Also, the rescue of blood glucose and HDL level was observed after the combined drug treatment, which might reduce adipose tissue dysfunction in visceral obesity (FIGS. 6H and 61). These results suggested that the combined drug treatment was effective in ameliorating the peripheral adiposity and inflammatory response in early-stage AD.

As to the AD pathology in the brain, it was found that the Aβ deposition and astrogliosis in the hippocampus of APP/PS1 mice were effectively mitigated by the combined drug treatment as revealed by Western blot and immunohistochemical analysis (FIGS. 6J and 6K). In addition, it was observed that autophagy-associated protein (p62, LC3), β-secretase (BACE), and Δ-secretase (Nicastrin) were markedly decreased (FIG. 6L).

These results on the preclinical pharmacology study indicated the feasibility of using the combined drug treatment that exert complementary effects on controlling lipid and glucose homeostasis to prevent or delay AD progression.

### Example 6: Population-Based Retrospective Analysis on Potential in Lowering Risk of Dementia

To assess the potential for applying the combined drugs identified in Example 5 for therapeutic application in patients with dementia, a long-term follow-up retrospective analysis of human efficacy predictions was conducted by using National Health Research Institutes Database (NHRID) of Taiwan. Specifically, a prospective matched cohort study was conducted to assess the effectiveness of the combined use of thyroid hormones and thiazolidinediones (TZDs) in reducing the risk of dementia, based on a dataset tracked 10,535 subjects with these two drugs for 10 years.

The results showed that the combined drugs significantly reduced the risk of getting dementia by 55% compared to 21.7% by triiodothyronine (T3) and 34.4% by TZD alone (Table 3).

TABLE 3
T3 and TZD reduce the incidence rate of dementia in Taiwan NHIRD database

| | Group 1 | | Group 2 | | Group 3 | |
|---|---|---|---|---|---|---|
| | TZD + T3 | Others | TZD + T3 | TZD only | TZD + T3 | T3 only |
| Number | 10,535 | 42,140 | 10,534 | 42,136 | 6,652 | 26,608 |
| Age, mean (S.D.) | 58.58 | 58.58 | 58.58 | 58.58 | 60.67 | 60.67 |
| | (11.46) | (11.46) | (11.46) | (11.46) | (11.70) | (11.70) |
| Male, n | 2,802 | 11,208 | 2,802 | 11,208 | 1,878 | 7,512 |
| (%) | (26.6%) | (26.6%) | (26.6%) | (26.6%) | (28.23%) | (28.23%) |
| Hypertension, n | 5,959 | 23,828 | 5,957 | 23,828 | 3,574 | 14,296 |
| (%) | (56.6%) | (56.6%) | (56.55%) | (56.55%) | (53.73%) | (53.73%) |
| Dyslipidemia, n | 4,504 | 18,012 | 4,503 | 18,012 | 3,078 | 12,312 |
| (%) | (42.75%) | (42.74%) | (42.75%) | (42.75%) | (46.27%) | (46.27%) |
| Incidence rate of | 7.28 | 11.21 | 7.29 | 8.85 | 8.44 | 10.17 |
| dementia (per | | | | | | |
| 1,000-person-year) | | | | | | |
| Relative risk | 0.446 | 1.00 | 0.783 | 1.00 | 0.656 | 1.00 |
| | | (Ref.)* | | (Ref.) | | (Ref.) |

*Ref.: comparison reference.

Since this result was obtained from a long-term follow-up retrospective analysis on prediction of drug efficacy in human population, it can be assumed that the pharmaceutical combination of thiazolidinediones with thyroid hormones might have the potential to improve clinical outcomes of dementia and be useful for early intervention of AD.

From the above, it is found that visceral adiposity acts as a dangerous event triggered by Aβ42 in early-stage AD to promote the brain pathogenesis, suggesting a fat-brain axis in early-stage AD. Based on these results, the present disclosure provides a combination therapy of an insulin sensitizer and a lipid metabolism modulator to restore metabolic homeostasis, thereby preventing or delaying AD progression. Accordingly, the pharmaceutical combination provided in the present disclosure effectively reduces early visceral adiposity and later brain pathology in AD, and can be used as an early intervention of AD.

It is obvious to a person skilled in the art that with the advancement of technology, the basic idea may be implemented in various ways. The embodiments are thus not limited to the examples described above; instead, they may vary within the scope of the claims.

The embodiments described hereinbefore may be used in any combination with each other. Several of the embodiments may be combined together to form a further embodiment. A method disclosed herein may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages.

All references and publications cited and discussed herein are hereby incorporated by reference in their entirety and to the same extent as if each reference or publication was individually incorporated by reference.

### REFERENCE

[1] Longo, M., et al. Adipose tissue dysfunction as determinant of obesity-associated metabolic complications. International Journal of Molecular Sciences 2019; 20(9):2358.

[2] Fessler, M. B. The challenges and promise of targeting the liver X receptors for treatment of inflammatory disease. Pharmacology and Therapeutics 2018; 181:1-12.

[3] Maczewsky, J., et al. Approved LXR agonists exert unspecific effects on pancreatic beta-cell function. Endocrine 2020; 68(3):526-535.

What is claimed is:

1. A pharmaceutical combination for preventing or treating a neurodegenerative disease in a subject in need thereof, comprising a first agent and a second agent, wherein the first agent is an insulin sensitizer and the second agent is a lipid metabolism modulator.

2. The pharmaceutical combination according to claim 1, wherein the insulin sensitizer is a hypoglycemic agent.

3. The pharmaceutical combination according to claim 2, wherein the hypoglycemic agent is selected from the group consisting of an agonist of peroxisome proliferator-activated receptor gamma (PPARγ), a sulfonylurea derivative, a biguanide derivative, a glucosidase inhibitor, and any combination thereof.

4. The pharmaceutical combination according to claim 3, wherein the PPARγ agonist is a thiazolidinedione derivative.

5. The pharmaceutical combination according to claim 4, wherein the thiazolidinedione derivative is selected from the group consisting of pioglitazone, rosiglitazone, troglitazone, lobeglitazone, ciglitazone, darglitazone, englitazone, netoglitazone, rivoglitazone, balaglitazone, and any combination thereof.

6. The pharmaceutical combination according to claim 3, wherein the sulfonylurea derivative is selected from the group consisting of glyburide, glibenclamide, glimepiride, chlorpropamide, glipizide, tolazamide, tolbutamide, and any combination thereof.

7. The pharmaceutical combination according to claim 3, wherein the biguanide derivative is selected from the group consisting of metformin, phenformin, buformin, and any combination thereof.

8. The pharmaceutical combination according to claim 3, wherein the glucosidase inhibitor is selected from the group consisting of acarbose, miglitol, voglibose, and any combination thereof.

9. The pharmaceutical combination according to claim 1, wherein the lipid metabolism modulator is an agonist of thyroid hormone receptor.

10. The pharmaceutical combination according to claim 9, wherein the agonist of thyroid hormone receptor is selected from the group consisting of triiodothyronine, thyroxine, an agonist of peroxisome proliferator-activated receptor alpha (PPARα), and any combination thereof.

11. The pharmaceutical combination according to claim 10, wherein the PPARα agonist is selected from the group consisting of clofibrate, gemfibrozil, ciprofibrate, bezafibrate, fenofibrate, and any combination thereof.

12. The pharmaceutical combination according to claim 1, which is formulated with at least one pharmaceutically acceptable carrier to form a transdermal patch.

13. A method for preventing or treating a neurodegenerative disease in a subject in need thereof, comprising:
administering to the subject a therapeutically effective amount of a first agent; and
administering to the subject a therapeutically effective amount of a second agent,
wherein the first agent is an insulin sensitizer and the second agent is a lipid metabolism modulator.

14. The method according to claim 13, wherein the first agent is a hypoglycemic agent.

15. The method according to claim 14, wherein the hypoglycemic agent is selected from the group consisting of an agonist of peroxisome proliferator-activated receptor gamma (PPARγ), a sulfonylurea derivative, a biguanide derivative, a glucosidase inhibitor, and any combination thereof.

16. The method according to claim 15, wherein the PPARγ agonist is a thiazolidinedione derivative.

17. The method according to claim 16, wherein the thiazolidinedione derivative is selected from the group consisting of pioglitazone, rosiglitazone, troglitazone, lobeglitazone, ciglitazone, darglitazone, englitazone, netoglitazone, rivoglitazone, balaglitazone, and any combination thereof.

18. The method according to claim 13, wherein the second agent is an agonist of thyroid hormone receptor.

19. The method according to claim 18, wherein the agonist of thyroid hormone receptor is selected from the group consisting of triiodothyronine, thyroxine, an agonist of peroxisome proliferator-activated receptor alpha (PPARα), and any combination thereof.

20. The method according to claim 19, wherein the PPARα agonist is selected from the group consisting of clofibrate, gemfibrozil, ciprofibrate, bezafibrate, fenofibrate, and any combination thereof.

21. The method according to claim 13, wherein the neurodegenerative disease is associated with accumulation of an amyloid β peptide in brain of the subject.

22. The method according to claim 13, wherein the neurodegenerative disease is mild cognitive impairment, early-stage Alzheimer's disease, vascular dementia, frontotemporal dementia, semantic dementia, or dementia with Lewy bodies.

23. The method according to claim 13, wherein the administrations of the first agent and the second agent reduce the accumulation of an amyloid β peptide in brain of the subject.

24. The method according to claim 13, wherein the administrations of the first agent and the second agent reduce visceral adiposity in the subject.

25. The method according to claim 13, wherein the first agent and the second agent are administered transdermally.
